(19) United States
(12) Patent Application Publication
(10) Pub. No.: US 2009/0069847 A1
(43) Pub. Date: Mar. 12, 2009

(54) SUTURE LOCK

(75) Inventors: KIYOSHI HASHIBA, Sao Paulo (BR); Brian K. Jones, Clemson, SC (US)

Correspondence Address: BRINKS HOFER GILSON & LIONE/CHICAGO/COOK, PO BOX 10395, CHICAGO, IL, 60610, US

(73) Assignee: WILSON-COOK MEDICAL INC., Winston-Salem, NC (US)

(21) Appl. No.: 12/191,001

(22) Filed: Aug. 13, 2008

Related U.S. Application Data
(60) Provisional application No. 60/956,575, filed on Aug. 17, 2007.

(51) Int. Cl. A61B 17/04

(52) U.S. Cl. 606/232

(57) ABSTRACT

A suture lock, as well as related methods, are provided for fixing strands of one or more sutures relative to tissue. The suture lock and method are simple and reliable in use, facilitate complete perforation closure and adjustment of the suture strands, and are adaptable to a variety of suture fixation and perforation closure situations. The suture lock includes a locking cylinder and a retaining sleeve. The locking cylinder has a tubular body defining an interior surface and an exterior surface. The interior surface defines a first interior passageway. The tubular body defines a first aperture and a second aperture that are spaced apart and in communication with the first interior passageway. The retaining sleeve defines a second interior passageway sized to receive the tubular body of the locking cylinder. The suture strands are compressed between the tubular body and the retaining sleeve.

### CROSS-REFERENCE TO RELATED APPLICATIONS

This application claims the benefit of U.S. Provisional Application Ser. No. 60/956,575, filed on Aug. 17, 2007, entitled “SUTURE LOCK”

### FIELD OF THE INVENTION

The present invention relates generally to suture locks for fixing the strands of one or more sutures relative to bodily tissue, such as for closing perforations in the tissue.

### BACKGROUND OF THE INVENTION

Perforations in the walls of internal organs and vessels may be naturally occurring, or formed intentionally or unintentionally. In order to permanently close these perforations and allow the tissue to properly heal, numerous medical devices and methods have been developed employing sutures, adhesives, clips, staples, anchors and the like. Many of these devices typically employ one or more sutures, the strands of which must be brought together and fixed in place in order to close the perforation.

Manually tying sutures strands together to close a perforation can be very complex and time consuming. For example, a significant level of skill and coordination is required by the medical professional, especially when the perforation and sutures are difficult to access within the body, such as in endoscopic or laparoscopic procedures. The numerous difficulties with manually tying sutures are well documented. In order to address these and other issues of manual suture tying, various automatic suture tying systems have been developed. Unfortunately, such automatic systems can be complex and difficult to use, and can be limited to use in certain procedures or situations.

### BRIEF SUMMARY OF THE INVENTION

The present invention provides a suture lock and related methods for fixing the strands of one or more sutures relative to tissue that is simple and reliable in use, facilitates complete perforation closure and adjustment of the suture strands, and that is adaptable to a variety of suture fixation and perforation closure situations. According to one embodiment of the suture lock, constructed in accordance with the teachings of the present invention, the suture lock generally includes a locking cylinder and a retaining sleeve. The locking cylinder has a tubular body defining an interior surface and an exterior surface. The interior surface defines a first interior passageway. The tubular body defines a first aperture and a second aperture that are spaced apart and in communication with the first interior passageway, and adapted for threading suture strands therebetween. The retaining sleeve defines a second interior passageway sized to receive the tubular body of the locking cylinder. The second interior passageway is sized to compress the suture strands between the tubular body and the retaining sleeve.

According to more detailed aspects of the suture lock, the first and second apertures are located between the ends of the locking cylinder. The suture strands extend along the exterior surface of the tubular body between the first and second apertures, and the suture strands are compressed between the exterior surface of the tubular body and the interior surface of the retaining sleeve when the second interior passageway receives the tubular body of the locking cylinder. The locking cylinder also includes a peripheral rim projecting from the tubular body that defines a shoulder sized and positioned to abut a distal end of the retaining sleeve. The distal end of the retaining sleeve is tapered, and preferably defines an end surface that is angled relative to a longitudinal axis of the retaining sleeve. The angled end surface facilitates the capture of the suture strands between the locking cylinder and the retaining sleeve. The locking cylinder and retaining sleeve compress the suture strands along sections of their length, the compressed sections extending between the first and second apertures.

According to another embodiment of the suture lock constructed in accordance with the teachings of the present invention, the suture lock generally includes a locking cylinder and a retaining sleeve. The locking cylinder has a tubular body defining a first interior passageway. The tubular body also defines a first aperture and a second aperture in communication with the first interior passageway. The retaining sleeve defines a second interior passageway sized to receive the tubular body of the locking cylinder. The suture lock is operable between an unlocked configuration and a locked configuration. The locking cylinder and retaining sleeve are connected in the locked configuration and are separated in the unlocked configuration. The locking cylinder and retaining sleeve compress elongate sections of the suture strands in the locked configuration. The elongate sections of the suture strands extend between the first and second apertures. According to further details, the suture strands extend through the first interior passageway, the first aperture, and the second aperture. The elongate sections of the suture strands are located outside of the tubular body in the locked configuration. The first and second apertures are sized and positioned to frictionally engage the suture strands with the locking cylinder when sufficient tension is placed on the suture strands.

A method for fixing strands of one or more sutures relative to tissue is also provided in accordance with the teachings of the present invention. According to the method, a suture lock is provided that generally includes a locking cylinder and a retaining sleeve, such as those described above. The suture strands are threaded through the locking cylinder. Specifically, the suture strands extend through the first internal passageway, through the first aperture, along the exterior of the tubular body, through the second aperture, and again through the first internal passageway. The locking cylinder is translated distally along the suture strands. The suture strands are threaded through the second internal passageway of the retaining sleeve, and the retaining sleeve is translated distally along the suture strands. The suture strands are placed in tension, and the retaining sleeve is translated over the locking cylinder to compress the suture strands between the retaining sleeve and locking cylinder.

According to more detailed aspects of the method, tension on the suture strands is maintained during the step of distally translating the locking cylinder. Tension on the suture strands is also maintained during the step of translating the retaining sleeve over the locking cylinder. The suture strands may be tensioned sufficiently to frictionally retain the locking cylinder at a desired position along the suture strands. The friction between the locking cylinder and suture strands may be overcome to adjust the position of the locking cylinder. Similarly, different tension may be placed on different suture strands, and the position of the locking cylinder adjusted. The locking cylinder and retaining sleeve are connected through their respective frictional engagement of the suture strands. When the tissue includes a perforation and the suture strands are connected to the tissue around the perforation, the step of placing the suture strands in tension includes substantially closing the perforation. The perforation is preferably closed against a distal end surface of the locking cylinder.

### BRIEF DESCRIPTION OF THE DRAWINGS

FIG. 1 is a perspective view of the locking cylinder constructed in accordance with the teachings of the present invention;

FIG. 2 is a cross-sectional view of the locking cylinder depicted in FIG. 1;

FIG. 3 is a perspective view of the locking cylinder depicted in FIG. 1, showing the locking cylinder in a locked configuration;

FIG. 4 is a cross-sectional view of the locking cylinder as depicted in FIG. 3; and

FIGS. 5 and 6 are cross-sectional views illustrating steps in a method of deploying the suture lock depicted in FIGS. 1-4.

### DETAILED DESCRIPTION OF THE INVENTION

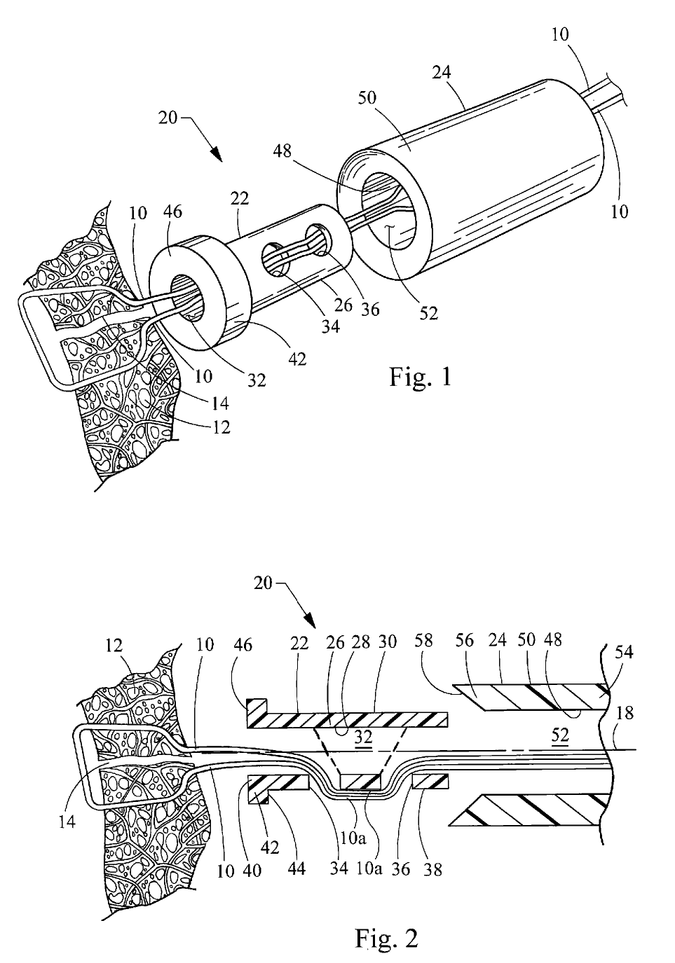

Turning now to the figures, FIGS. 1 and 2 depict a suture lock 20 constructed in accordance with the teachings of the present invention. The suture lock 20 generally includes a locking cylinder 22 and a retaining sleeve 24 that are used to fix the strands 10 of one or more sutures relative to bodily tissue 12, such as for closing a perforation 14 in the tissue 12. Generally, the suture strands 10 will be placed through the tissue 12 and connected thereto using standard suturing techniques or devices such as T-anchors, staples or the like, leaving the strands 10 on one side (e.g., the proximal side) of the tissue 12 for tying them together. Although the locking cylinder 22 and retaining sleeve 24 have been depicted as having circular cross-sections, it will be recognized that other cross-sectional shapes may be used including oval, square, etc. The locking cylinder 22 generally defines a longitudinal axis 18, which is also generally parallel to the longitudinal axis of the retaining sleeve 24 when it is interconnected with the locking cylinder 22.

The locking cylinder 22 generally comprises a tubular body 26 having an interior surface 28 and an exterior surface 30. The tubular body 26 and its interior surface 28 define a first interior passageway 32. A first aperture 34 and a second aperture 36 are formed in the tubular body 26 and extend from the exterior surface 30 to the interior surface 28. The first and second apertures 34, 36 are longitudinally spaced apart, although it will be recognized by those skilled in the art that the first and second apertures 34, 36 may be circumferentially spaced apart, or both longitudinally and circumferentially spaced apart. The first and second apertures 34, 36 are located between the proximal end 38 and the distal end 40 of the locking cylinder 22. The distal end 40 of the locking cylinder 22 also includes a peripheral rim 42 projecting radially from the tubular body 26 and defining a shoulder 44 for abutting against the retaining sleeve 24, as will be described further hereinbelow.

The retaining sleeve 24 generally includes an interior surface 48 and an exterior surface 50. The interior surface 48 defines a second interior passageway 52 that is sized to receive the tubular body 26 of the locking cylinder 22 therein. The retaining sleeve 24 includes a proximal end 54 and a distal end 56. The distal end 56 is preferably tapered, such as a chamfer, and defines an end surface 58 that is angled (preferably less than 90 degrees) relative to the longitudinal axis 18 of the retaining sleeve 24. The angled end surface 58 prevents interference when the locking cylinder 22 is inserted through the retaining sleeve 24, and facilitates the capture of the suture strands 10 between the locking cylinder 22 and sleeve 24.

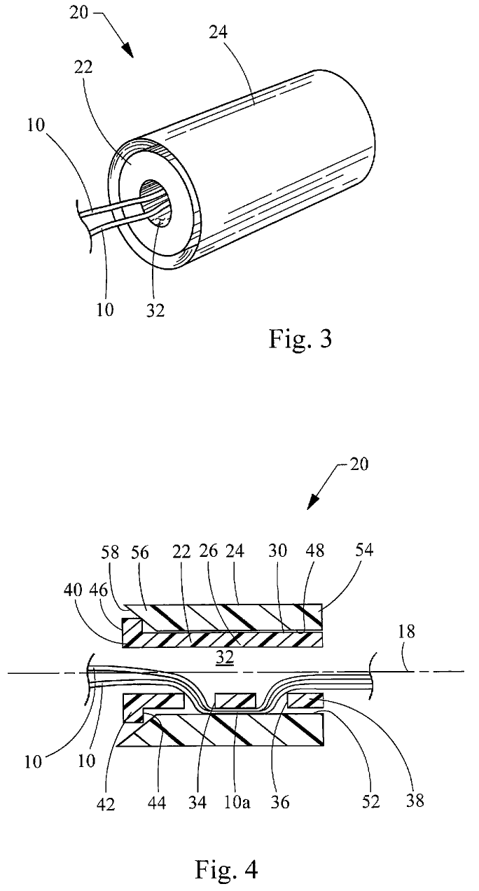

FIGS. 1 and 2 depict the suture lock 20 in an unlocked configuration, while FIGS. 3 and 4 depict the suture lock 20 in a locked configuration. Generally, the locking cylinder 22 and retaining sleeve 24 are separated in the unlocked configuration, and are connected in the locked configuration. When locked, the retaining sleeve 24 concentrically receives the locking cylinder 22 within its interior passageway 52, and the distal end 56 is proximate the distal end 40 and its shoulder 44. The locking cylinder 22 and retaining sleeve 24 may be directly frictionally engaged together, and/or are connected through their respective frictional engagement of the suture strands 10 in the locked configuration, as described further below.

The suture strands 10 extend from the tissue 12 to the locking cylinder 22, through the first interior passageway 32, through the first aperture 34, along the exterior surface 30 of the tubular body 26, through the second aperture 36, and again through the first interior passageway 32. As best seen in FIG. 4, the size of the second interior passageway 52 relative to the tubular body 26 is such that the suture strands 10 are compressed between the exterior surface 30 of the tubular body 26 and the interior surface 48 of the retaining sleeve 24. Specifically, elongate sections 10a of the suture strands 10 travel along the exterior surface 30 of the tubular body 26 between the first aperture 34 and the second aperture 36. These elongate sections 10a of the suture strands 10 are compressed between the locking cylinder 22 and retaining sleeve 24 in the locked configuration. That is, the locking cylinder 22 and retaining sleeve 24 compress the suture strands 10 along elongate sections 10a. The elongate sections 10a of the suture strands 10 may or may not undergo plastic deformation, which can facilitate locking of the medical device 20. The elongate sections 10a of the suture strands 10 extend between the first and second apertures 34, 36, and are generally located outside of the tubular body 26 of the locking cylinder 22 in the locked configuration.

It will be recognized that the locking cylinder 22 may have a discontinuous first interior passageway 32. For example, the cylinder 22 may be solid between the first and second apertures 34, 36, or may have cross members or other elements extending across the interior passageway 32 (as shown by the dotted lines in FIG. 2. In such embodiments the first aperture 34 would be in communication with a first portion of the interior passageway 32 while the second aperture 36 would be in communication with a second portion of the interior passageway 32. Thus, as used herein, an interior passageway may have portions which are not in communication with each other. When the cylinder 22 has such a solid portion or other crossing members, these structures can define guide surfaces which are structured to guide the suture 10 towards or away from (depending on the direction of threading the suture) the interior passageway 32, the first and second apertures 34, 36 and the open ends of the locking cylinder 22.

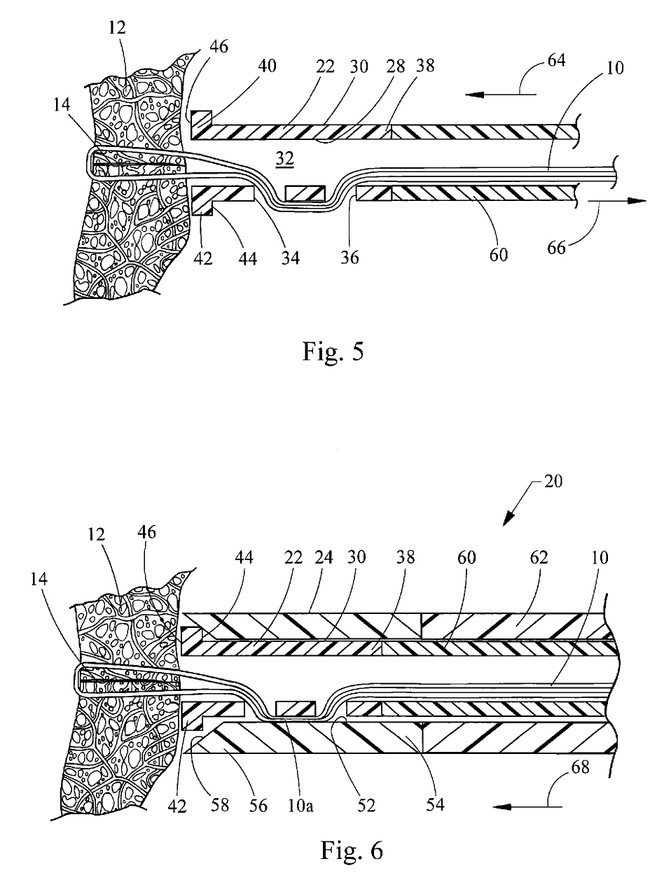

Turning now to FIGS. 5 and 6, a method for fixing the suture strands 10 relative to the tissue 12 will now be described. A suture lock such as the suture lock 20 previously described is provided. Proximal ends of the suture strands 10 (not shown, the proximal ends normally being located outside of the patient in endoscopic and laparoscopic procedures) are threaded through the locking cylinder 22. In particular, the suture strands are placed through the first interior passageway 32 adjacent the distal end 40 of the locking cylinder 22, then through the first aperture 34, along the exterior surface 30 of the tubular body 26, through the second aperture 36, and again through the first interior passageway 32 adjacent the proximal end 38 of the locking cylinder 22.

A first pushing catheter 60 is used to distally translate the locking cylinder 22 along the suture strands 10, as indicated by arrow 64 in FIG. 5. The pushing catheter 60 may take the form of any catheter or cannula known in the art, but preferably has sufficient strength and rigidity for longitudinal force transmission, while still providing flexibility for navigation of a patient's body. Exemplary pushing catheters are sold by Cook Medical and Cook Endoscopy. The pushing catheter 60 may be loosely press-fit to the proximal end 38 of the locking cylinder 22 or may simply abut the proximal end 38 for longitudinal force transmission, as shown. It will also be recognized by those skilled in the art that other devices for translating the locking cylinder 22 along the suture strands 10 may be employed, such as wire guides, pushing rods, and the like, or even by hand during open surgery.

Notably, the proximal ends of the suture strands 10 may be pulled in a proximal direction in order to place the suture strands 10 in tension, as indicated by arrow 66 in FIG. 5. When sufficiently tensioned, the suture strands 10 frictionally engage the locking cylinder 22 to retain the locking cylinder 22 at a desired position along the suture strands 10. That is, the suture strands 10 follow the somewhat tortuous path through the first interior passageway 32 and the first and second apertures 34, 36, and thus sufficient tension causes the suture strands 10 to frictionally engage the locking cylinder 22 within the apertures 34, 36 and along the interior surface 28 and the exterior surface 30 of the locking cylinder 22. This allows the medical professional to facilitate translation or restrict translation of the locking cylinder 22 simply by reducing the tension or increasing the tension on the suture strands 10, respectively. At the same time, the friction between the suture strands and the locking cylinder 22 may be overcome at any time with sufficient force placed on the locking cylinder 22, allowing translation and adjustment of the locking cylinder 22 even when the suture strands 10 are in tension.

With the suture strands 10 in tension, the locking cylinder 22 is translated distally to a position proximate the tissue 12, as shown in FIG. 5. The suture strands 10 are sufficiently tensioned to frictionally retain the locking cylinder 22 at a desired position. When a perforation 14 is present in the tissue 12, the suture strands 10 are tensioned sufficiently to substantially close the perforation 14 in the tissue 12. The position of the locking cylinder 22 may be adjusted as desired, and the tension on individual suture strands 10 may be adjusted to guide or otherwise facilitate the same.

The suture strands 10 are also threaded through the retaining sleeve 24, and in particular through the second interior passageway 52. As best seen in FIG. 6, a second pushing catheter 62 is used to distally translate the retaining sleeve 24 along the suture strands 10, as indicated by arrow 68. The second pushing catheter 62 may have a construction similar to the first pushing catheter 60 described above, or any of its variations or alternatives. As the retaining sleeve 24 approaches the locking cylinder 22, the angled end surface 58 of the retaining sleeve 24 helps capture the elongate sections 10a of the suture strands 10 between the exterior surface 30 of the locking cylinder 22 and the interior surface 48 of the retaining sleeve 24.

As the retaining sleeve 24 is distally translated over the tubular body 26 of the locking cylinder 22, the elongate sections 10a of the suture strands 10 are compressed, and the locking cylinder 22 and retaining sleeve 24 are connected through their respective frictional engagement of the suture strands 10. The tension on the suture strands 10 is preferably maintained while the retaining sleeve 24 is translated over the locking cylinder 22. The relative positions of the locking cylinder 22 and retaining sleeve 24 are limited by the peripheral rim 42 and shoulder 44 of the locking cylinder 22, which abuts the distal end 56 of the retaining sleeve 24 as shown. Preferably, the perforation 14 in the tissue 12 is closed against a distal end surface 46 of the locking cylinder 22. To release the suture lock 20, the suture strands 10 may be cut, or the first pushing catheter 60 may be used to hold the locking cylinder 22 while the retaining sleeve 24 is grasped (such as with a snare, forceps or similar device) and physically withdrawn against the friction of the suture strands 10.

It will be recognized by those skilled in the art that prior to interconnection of the locking cylinder 22 and retaining sleeve 24, the tension in the suture strands 10 may be modified to facilitate adjustment of the position of the locking cylinder 22. Similarly, different tension may be placed on different suture strands 10, and the position of the locking cylinder 22 adjusted accordingly. The components of the suture lock 20 may be constructed of many materials, such as stainless steel, titanium, nitinol or other metals/alloys, as well as various ceramics or plastics such as polycarbonates (PC), polyamides including Nylon(™). polytetrafluorethylenes (i.e. PTFE and EPTFE), polyethylene ether ketones (PEEK), polyvinylchlorides (PVC), polyimides, polyurethanes, and polyethylenes (high, medium or low density), including multi-layer or single layer constructions with or without reinforcement wires, coils or filaments.

Preferably, the method is performed under direct visualization, such as through the use of an endoscope or other fiber optic-based visualization systems including catheter-based systems. As such, positioning of the locking cylinder 22 may be viewed and adjusted. In some situations, the size of the suture lock 20 and the pushing members (e.g. catheters 60, 62) are such that the suture lock system may be employed through the working channel of an endoscope, although the system may also be employed in parallel with an endoscope or other visualization system. Thus the pushing members, and preferably the suture lock, are sized for delivery through the working channel of an endoscope. It will also be recognized that other visualization techniques, such as ultrasound, fluoroscopy and the like may be employed in conjunction with the devices and methods of the present invention. For example roughing certain portions of the suture lock 20 may be ideal for use with an ultra-sound capable endoscope. Finally, an appropriate handle or actuator for controlling the relative translation of the pushing members such as the pushing catheters 60, 62 will be readily envisioned by one of ordinary skill in the art.

While the disclosed suture locks and methods are applicable to a variety of suturing situations, the suture locks are especially useful in closing perforations in internal bodily walls, such as those formed in transluminal procedures. In such procedures, a perforation or opening is formed in the wall of one lumen, say the gastric wall, and a medical device such as an endoscope is placed through that opening for access to an adjacent structure or cavity, such as the peritoneal cavity. While there are many benefits of such minimally invasive procedures, complete and proper closure of the opening is essential to the success of such procedures. The suture locks and methods of the present invention are particularly well suited for closing these perforations since they are simple and reliable in use, and are adaptable to a variety of suture fixation and perforation closure applications. For example, any number of sutures and their suture strands may be employed, and the relative sizes of the locking cylinder and retaining sleeve may be adjusted based on suture size, perforation size and the like. Superior control over the positioning of the suture lock is provided, while at the same time providing a device or method that is easy to use and deploy. The suture locks may be used in open surgery, endoscopically, laparoscopically, or in other minimally invasive interventional procedures.

The foregoing description of various embodiments of the invention has been presented for purposes of illustration and description. It is not intended to be exhaustive or to limit the invention to the precise embodiments disclosed. Numerous modifications or variations are possible in light of the above teachings. The embodiments discussed were chosen and described to provide the best illustration of the principles of the invention and its practical application to thereby enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly, legally, and equitably entitled.

1. A suture lock for fixing strands of one or more sutures relative to tissue, the suture lock comprising:
a locking cylinder having a tubular body defining an interior surface and an exterior surface, the interior surface defining a first interior passageway, the tubular body defining a first aperture and a second aperture, the first aperture spaced apart from the second aperture, the first aperture and the second aperture in communication with the first interior passageway and adapted for threading suture strands therebetween; and
a retaining sleeve defining a second interior passageway sized to receive the tubular body of the locking cylinder, the second interior passageway sized to compress the suture strands between the tubular body and the retaining sleeve.

2. The suture lock of claim 1, wherein the first and second apertures are located between the ends of the locking cylinder.

3. The suture lock of claim 1, wherein the suture strands are compressed between the exterior surface of the tubular body and the interior surface of the retaining sleeve when the second interior passageway of the retaining sleeve receives the tubular body of the locking cylinder.

4. The suture lock of claim 3, wherein the suture strands are pressed against the exterior surface of the tubular body between the first and second apertures.

5. The suture lock of claim 1, wherein the suture lock is operable between an unlocked configuration and a locked configuration, the locking cylinder and retaining sleeve being connected in the locked configuration, and wherein the suture strands extend through the first interior passageway, the first aperture, and the second aperture, a section of the suture strands being located outside of the tubular body and compressed against the exterior surface of the tubular body in the locked configuration.

6. The suture lock of claim 1, wherein the locking cylinder includes a peripheral rim projecting from the tubular body, the peripheral rim defining a shoulder sized and positioned to abut a distal end of the retaining sleeve.

7. The suture lock of claim 1, wherein a distal end of the retaining sleeve is tapered.

8. The suture lock of claim 1, wherein the distal end defines an end surface angled relative to a longitudinal axis of the retaining sleeve.

9. The suture lock of claim 1, wherein the locking cylinder and the retaining sleeve compress the suture strands along sections of their length, the compressed sections of the suture strands extending between the first and second apertures.

10. A suture lock for fixing strands of one or more sutures relative to tissue, the suture lock comprising:
a locking cylinder having a tubular body defining a first interior passageway, the tubular body defining a first aperture and a second aperture, the first aperture and the second aperture in communication with the first interior passageway;
a retaining sleeve defining a second interior passageway sized to receive the tubular body of the locking cylinder; and
the suture lock being operable between an unlocked configuration and a locked configuration, the locking cylinder and retaining sleeve being connected in the locked configuration and being separated in the unlocked configuration, the locking cylinder and retaining sleeve compressing elongate sections of the suture strands between the first and second apertures in the locked configuration.

11. The suture lock of claim 10, wherein the suture strands extend through the first interior passageway, the first aperture, and the second aperture, and wherein the elongate sections of the suture strands are located outside of the tubular body in the locked configuration.

12. The suture lock of claim 11, wherein first and second apertures are sized and positioned to frictionally engage the suture strands with the locking cylinder when sufficient tension is placed on the suture strands.

13. The suture lock of claim 10, wherein the locking cylinder and retaining sleeve are sized for delivery through a working channel of an endoscope.

14. A method for fixing strands of one or more sutures relative to tissue, the method comprising:
providing a suture lock comprising a locking cylinder and a retaining sleeve, the locking cylinder having a tubular body defining a first interior passageway, the tubular body defining spaced apart first and second apertures in communication with the first interior passageway, the retaining sleeve defining a second interior passageway sized to receive the locking cylinder and compress the suture strands between the tubular body and the retaining sleeve;
threading the suture strands through the locking cylinder, the suture strands extending through the first internal passageway, through the first aperture, along the exterior of the tubular body, through the second aperture, and again through the first internal passageway;
translating the locking cylinder distally along the suture strands;
threading the suture strands through the second internal passageway of the retaining sleeve;
translating the retaining sleeve distally along the suture strands;
placing the suture strands in tension; and
translating the retaining sleeve over the locking cylinder to compress the suture strands between the retaining sleeve and locking cylinder.

15. The method of claim 14, further comprising the step of maintaining tension on the suture strands during the step of translating the locking cylinder distally.

16. The method of claim 15, wherein the suture strands are tensioned sufficiently to frictionally retain the locking cylinder at a desired position along the suture strands.

17. The method of claim 16, further comprising the step of overcoming the friction between the suture strands and the locking cylinder, and adjusting the position of the locking cylinder.

18. The method of claim 16, further comprising the steps of placing different tension on different suture strands and adjusting the position of the locking cylinder.

19. The method of claim 14, further comprising the step of maintaining tension on the suture strands during the step of translating the retaining sleeve over the locking cylinder.

20. The method of claim 14, the tissue including a perforation and the suture strands connected to the tissue around the perforation, wherein the step of placing the suture strands in tension includes substantially closing the perforation against a distal end surface of the locking cylinder.
